(19) United States
(12) Patent Application Publication
(10) Pub. No.: US 2007/0088273 A1
(43) Pub. Date: Apr. 19, 2007

(54) Method and apparatus for intravascular cannulation

(75) Inventor: Ahmad N. Rafi, Little Rock, AR (US)

Correspondence Address: Kyla D. Cummings;SPEED LAW FIRM, Suite 1200, 111 Center Street, Little Rock, AR, 72201, US

(73) Assignee: Ahmad N. Rafi

(21) Appl. No.: 11/507,713

(22) Filed: Aug. 22, 2006

Related U.S. Application Data
(60) Provisional application No. 60/710,052, filed on Aug. 22, 2005.

(51) Int. Cl. A61M 5/178

(52) U.S. Cl. 604/164.01

(57) ABSTRACT

A method and apparatus for intravascular cannulation in which a distal assembly of intravascular catheter and needle improves simultaneous placement of the needle (inner metallic cannula) and the catheter (outer plastic cannula) in the vessel lumen. The distal elliptical aperture of the needle lumen is located in both the beveled and non-beveled parts of the needle to either side of the bevel take-off point. The distal bevel end is mostly non-cannulated and serves primarily to puncture the skin and blood vessel. The distal annular end of the catheter has a half oval opening in one side of the annular ring. The half oval opening of the catheter overlies and coincides with the oval non-beveled part of the distal aperture of the needle when assembled together. The distal end of the catheter has a rounded spur on the opposite side facing the half oval opening. When assembled together, the rounded spur of the catheter extends beyond the distal margin of the aperture in the needle on the side opposite to the bevel. The needle has a colored marking (e.g., blue, green or white) proximally to indicate the position of the bevel tip on withdrawal when it is just within the safe confines of the distal opening of the catheter.

### CROSS-REFERENCE TO RELATED APPLICATIONS

This application claims benefit of U.S. provisional patent application Ser. No. 60/710,052, filed Aug. 22, 2005, which is herein incorporated by reference in its entirety.

### BACKGROUND OF THE INVENTION

Intravascular cannulation is the most commonly performed procedure in any medical facility all over the world. The typical prior art intravascular cannula comprises an outer plastic cannula (the catheter) and an inner metallic cannula (the needle). The distal end of the needle is beveled to facilitate puncture of the skin and blood vessel. The lumen of the needle ends in an elliptical aperture which is housed within the bevel of the needle. Proximally, the needle has a transparent hub base (the flash chamber) to allow visual observation of the “flashback” (e.g., of blood in the case of a blood vessel). The distal end of the catheter is non-beveled with an annular opening and it ends just proximal to the metallic bevel “take off” point (the bevel heel). The size and length of a bevel increases proportionally as the cannula gauge increases.

During routine intravascular cannulation, a health care worker inserts and moves the cannula towards the intended blood vessel. On flashback of blood in the flash chamber, the user assumes that the distal end of the cannula is within the vessel lumen. At this point, the user advances the catheter over the needle. The process of intravascular cannulation is complete when the needle is withdrawn while leaving the catheter inside the vessel lumen.

There are problems with the design of the distal assembly of commercially available intravascular cannulas. Due to these problems, there is some degree of technique failure in intravascular cannulation even in experienced hands. Failure rate is much higher in a novice user. In the opinion of the present inventor, intravascular cannulation is such a simple technique that the success rate should be no less than 100% in all hands barring some failure owing to pathology of the vessel itself. Also, there is the common observation that the success rate is higher for smaller gauge cannulas than large gauge cannulas. That is why many practitioners tend to use smaller gauge cannulas even when the clinical situation demands placement of larger gauge cannulas. The present inventor believes that these problems may be solved by changing the design of a typical cannula's distal assembly.

In the prior art cannula assembly, the catheter is shorter than the needle and ends proximal to the bevel take-off point. During placement, when a practitioner observes “flashback” of blood in the proximal hub, the bevel of the needle is assumed to be within the vessel lumen. At this point, there are three possibilities so far as the position of the distal end of the catheter is concerned:

Possibility 1: The distal end of the catheter is within the vessel lumen, which is the ultimate aim of intravascular cannulation.

Possibility 2: The distal end of the catheter is within the vessel wall structures.

Possibility 3: The distal end of the catheter is outside the vessel wall.

Possibilities 2 and 3 are responsible for most of the technique failures observed with intravascular cannulation. This also explains the high failure rate with large size cannulas as the size and length of the bevel (distal to end of the catheter) increase with the gauge of the cannula, thereby increasing the likelihood of possibilities 2 and 3. In essence, the “flashback” of blood in the flash chamber is no guarantee of cannulation success and any health care worker would testify to the validity of the statement. In the inventor's opinion, the “flashback” should be a sure sign of cannulation success, which could be achieved by changing the design of the distal cannula assembly.

### SUMMARY OF THE INVENTION

The present invention solves the problems of the prior art by making certain modifications in the distal assembly of an intravascular cannula with an aim to improve simultaneous placement of the needle and the catheter in the vessel lumen:

1. The distal elliptical aperture of the needle lumen is located only partially in the bevel of the needle (See FIGS. 2A-C).

2. The distal aperture of the needle lumen is located partly proximally and partly distally to the bevel take-off point (See FIGS. 2A-C).

3. The distal bevel is mostly non-cannulated and serves primarily to puncture the skin and blood vessel (See FIGS. 2A-C).

4. The distal annular end of the catheter has a half oval (included within the meaning of half oval opening is a half circle opening) opening in one side of the annular ring (See FIGS. 3A-B).

5. The half oval opening of the catheter overlies and coincides with the non-bevel part of the distal aperture of the needle when assembled together (See FIGS. 5A-C).

6. The distal end of the catheter has a round spur on the opposite side facing the half oval opening (See FIGS. 4A-C).

7. When assembled together, the rounded spur of the catheter extends beyond the distal margin of the aperture in the needle on the side opposite to the bevel (See FIG. 5A).

the needle has a colored marking; e.g., blue, green or white, proximally to indicate the position of the bevel tip on withdrawal when it is just within the safe confines of the distal opening of the catheter (See FIG. 6).

The objectives to be achieved with the present invention are to have one intravascular cannula that can be used for both intravenous and intra-arterial access with equal success, in which “flashback” is a definite sign of perfect cannulation, in which success does not depend on the gauge of the cannula and where there is standardization of the procedure with a consistent approach to the technique.

### BRIEF DESCRIPTION OF THE DRAWINGS

So that the manner in which the above-recited features of embodiments of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is an enlarged side view of a prior art intravascular cannula.

FIG. 2A is a top view of the needle of the present invention. FIG. 2B is a side view of the needle of FIG. 2A. FIG. 2C is a side cross-sectional view of the needle of FIG. 2A taken along a longitudinal axis of the needle.

FIG. 3A is a side view of the catheter of the present invention. FIG. 3B is a top view of the catheter of FIG. 3A.

FIG. 4A is a view of catheter of the present invention showing a rounded spur on the distal end of the catheter. FIG. 4B is a top view of the catheter of FIG. 4A. FIG. 4C is a bottom view of the catheter of FIG. 4A.

FIG. 5A is a side view of the assembled needle and catheter of the present invention. FIG. 5B is a top view of the assembled needle and catheter of FIG. 5A. FIG. 5C is a bottom view of the assembled needle and catheter of FIG. 5A.

FIG. 6 is a side view of the embodiment of FIGS. 5A-C showing the needle withdrawn within the distal opening of the catheter.

FIGS. 7A-C are sectional views of a blood vessel showing stations of a typical prior art intravascular cannula during placement. FIG. 7A illustrates Possibility 1, FIG. 7B illustrates Possibility 2, and FIG. 7C illustrates Possibility 3 as described herein.

FIGS. 8A-F show the relationship between the distal aperture of the needle and the distal opening of the catheter. FIG. 8A is a side view of the needle. FIG. 8B is a top view of the needle showing the face of the bevel of the needle. FIG. 8C is a top view of the catheter. FIG. 8D is a side view of the catheter. FIG. 8E is a top view of the cannula assembled from the needle and catheter. FIG. 8F is a side view of the cannula of FIG. 8E.

### DETAILED DESCRIPTION

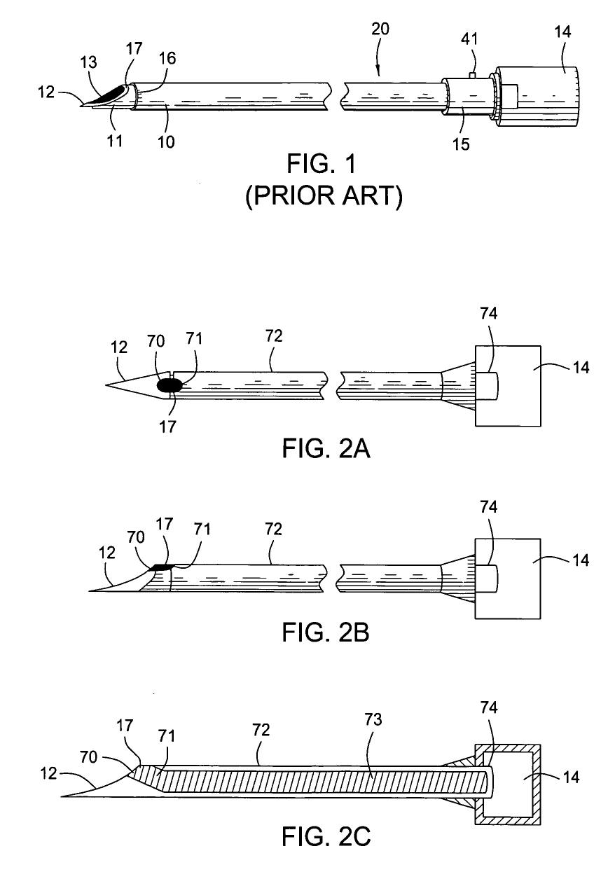

In the prior art as shown in FIG. 1, a common intravascular cannula comprises an outer plastic cannula described herein as a catheter 10 and an inner metallic cannula described herein as a needle 11 (The entire assembly of a catheter 10 and a needle 11 is described herein as an intravascular cannula 20). The distal end of the needle 11 has a bevel 12 to facilitate puncture of skin and blood vessel. The lumen of the needle 11 ends in an elliptical aperture 13 which is housed within the bevel 12. Proximally, the needle 11 has a transparent hub base 14 (also described herein as a “flash chamber”) to allow visual observation of the “flashback” of blood. The proximal end 15 of the catheter 10 is adjacent to the hub base 14. The distal end of the catheter 10 is non-beveled with an annular opening 16 and ends just proximally to the bevel take-off point 17 (also called herein the “bevel heel”). The size and length of a bevel 12 increases proportionally as the gauge of the intravascular cannula 20 increases. During routine intravascular cannulation, a health care worker inserts and moves the intravascular cannula 20 towards the intended blood vessel. On flashback of blood in the hub base 14, the practitioner assumes that the cannula tip is within the vessel lumen. At this point, the practitioner advances the catheter 10 over the needle 11. The process of intravascular cannulation is complete when the needle 11 is withdrawn while leaving the catheter 10 inside the vessel lumen.

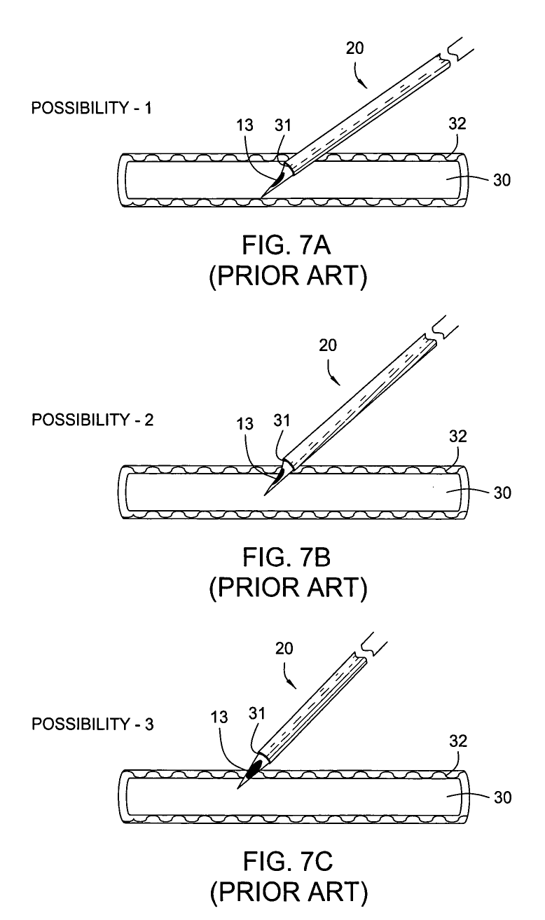

In the prior art intravascular cannula 20, the catheter 10 is shorter than the needle 11 and ends proximal to the bevel take-off point 17. During placement, when a practitioner observes “flashback” of blood in the hub base 14, the elliptical aperture 13 of the bevel 12 of the needle 11 is assumed to be within the vessel lumen 30. At this point, there are three possibilities as shown in FIGS. 7A-C, so far as the position of the distal end 31 of the catheter 10 is concerned:

Possibility 1: As shown in FIG. 7A, the distal end 31 of the catheter 10 is within the vessel lumen 30, which is the ultimate aim of intravascular cannulation.

Possibility 2: As shown in FIG. 7B, the distal end 31 of the catheter 10 is within the vessel wall 32.

Possibility 3: As shown in FIG. 7C, the distal end 31 of the catheter 10 is outside the vessel wall 32.

During routine intravascular placement, once the “flashback” is observed, the user is supposed to hold the needle 11 still (motionless) while only moving the catheter 10 over it into the targeted vessel lumen 30. The intent is to advance the catheter 10 into the blood vessel lumen 30 in case its distal end 31 and the distal elliptical opening 13 of the needle 11 are within the structures of the vessel wall 32 or outside the vessel wall 32. (See Possibilities 2 and 3—FIGS. 7B and C, respectively). The observation of the present inventor is that most practitioners do not follow the correct technique of intravascular cannulation. Once the “flashback” is observed, instead of holding the needle 11 still while moving the catheter 10 over it, most practitioners tend to withdraw the needle 11 back into the catheter 10 and then advance the catheter 10 or the whole intravascular cannula 20 into the vessel lumen 30. In Possibility 1, that leads to no problem (See FIG. 7A). In Possibility 2 and Possibility 3 (FIGS. 7B and C), this maneuver leads to advancement of the catheter 10 into the structures of the vessel wall 32 or outside the vessel wall 32 as the bevel 12 of the needle 11 has already been withdrawn (backward) inside the distal end 31 of the lumen of the catheter 10. With experience, after the moment of “flashback”, many practitioners learn to advance the intravascular cannula 20 a little more onward before pulling back the bevel 12 into the catheter 10. This is how they tend to avoid the problems accruing from Possibility 2 and Possibility 3 (FIGS. 7B and C).

The preferred embodiment of the present invention makes use of this common error in the technique of intravascular cannulation. As shown if FIGS. 2A-C, the distal aperture 13 of the needle 11 is moved away from the bevel 12 to the bevel take-off point 17 so that the aperture 13 comprises a bevel part 70 and a non-bevel part 71. The distal aperture 13 is located partially rather than entirely in the bevel 12. The non-bevel part 71 of the aperture 13 is located in the cylindrical wall of the body 72 of the needle 11 just proximal to the bevel take-off point 17. The bevel part 70 of the aperture 13 is located in the bevel 12 just distal to the bevel take-off point 17. Both the non-bevel part 71 and the bevel part 70 of the distal aperture 13 form one single orifice which serves as the distal end of the lumen 73 of the needle 11 which communicates to the proximal end 74 of the needle 11 and into the transparent hub base 14. The non-bevel part 71 of the distal aperture 13 is in the shape of a half oval (the meaning of “half-oval” embraces a “half circle”). The bevel take-off point 17 is the geometrical center of the half oval of the non-bevel part 71. The bevel part 70 of the distal aperture 13 covers one-third or less of the face of the bevel 12. In this embodiment, the distal two-thirds or more of the bevel 12 is solid and therefore non-cannulated and serves only to puncture the skin and blood vessel.

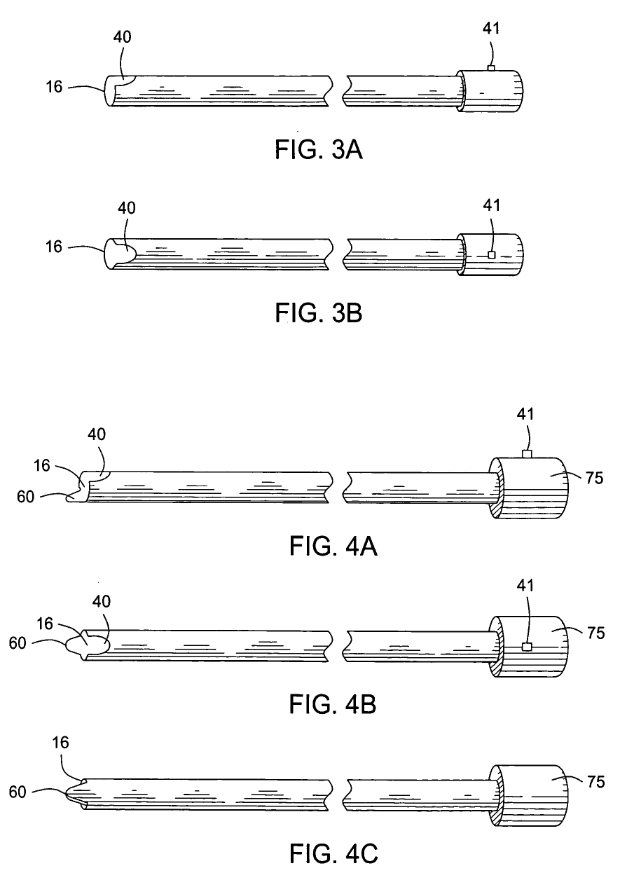

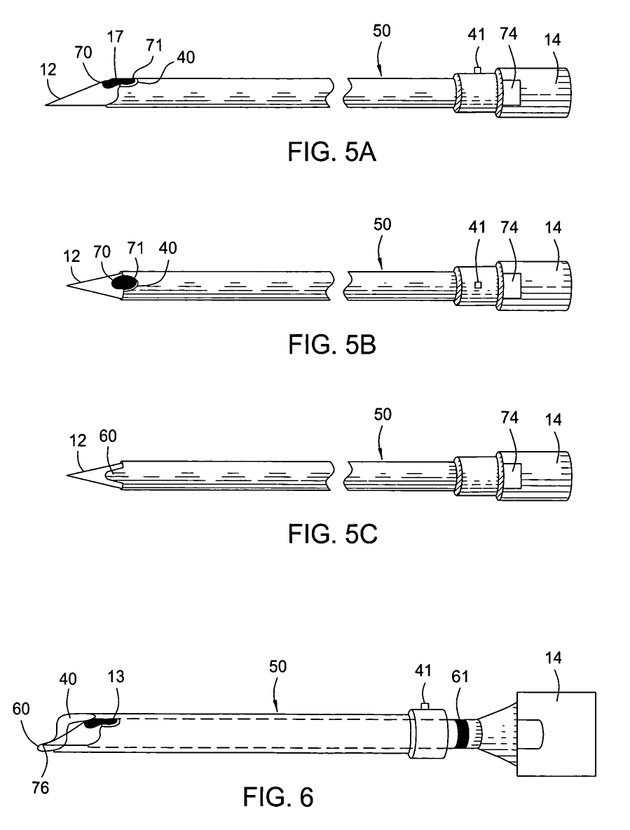

The distal annular opening 16 of the catheter 10 as shown in FIGS. 3A-B, ends art the bevel take-off point 17 as shown in FIG. 5A. The proximal end 75 of the catheter is provided with a Luer lock. On the annular opening 16 at the distal end 31 if the catheter 10, a half oval opening 40 is made on one side of the annular opening 16. The half oval opening 40 overlies and corresponds with the non-bevel part 71 of the distal aperture 13 of the needle when the catheter 10 and needle 11 are assembled together as a complete intravascular cannula 50. The assembly of the intravascular cannula 50 is achieved through axial and circumferential alignment by means provided at the proximal ends of the catheter and needle. A pointer 41 on the proximal end of the catheter 10 indicates the side of the bevel 12 when the catheter 10 and the needle 11 are assembled together. A cannula assembly 50 of the present invention is shown is FIGS. 5A-c. This arrangement makes sure that the distal end of the catheter 10 is inside the vessel lumen 30 precisely at the moment when the “flashback” is observed. The problems related to Possibility 2 and Possibility 3 are clearly eliminated. The downside is that a significant length of the distal cannula assembly 50 (including the non-cannulated portion of the bevel 12 and the two corresponding openings 13, 40) has to be within the vessel lumen 30. This would increase the possibility of counter-puncture by the sharp end of the bevel 12. In this scenario, advancing the catheter 10 over the needle 11 (the usual recommendation for common intravascular cannulas) could lead to placement of the catheter 10 out of the blood vessel via the counter-puncture. This situation, though, could be rectified by pulling back the catheter 10 into the vessel lumen 30. The inventor, however, recommends pulling out the bevel 12 backward into the catheter 10, once the “flashback” is observed in FIG. 6. Thereafter, the catheter 10 is safely advanced into the vessel lumen 30 without any possibility of inadvertent placement via the counter-puncture.

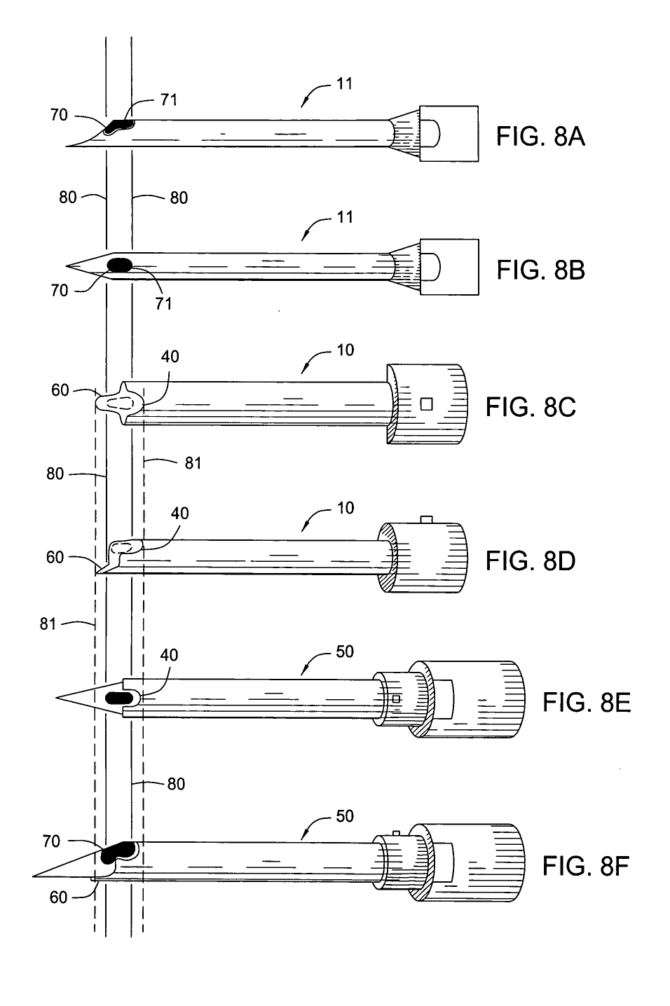

However, what if the corresponding openings 13,40 in the intravascular cannula assembly 50 are halfway inside the vessel lumen 30 and halfway in the structures of the vessel wall 32 or outside the vessel wall as discussed in Possibility 2 and Possibility 3? This situation is rectified as shown in FIGS. 4A-C by having a rounded spur 60 on the distal end of the catheter 10 and opposite to the openings 13, 40. The round spur 60 extends beyond the distal margin of the annular opening 16 of the catheter 10. In the assembly of the intravascular catheter 50, the rounded spur 60 extends beyond the distal margin of the aperture 70 of the bevel 12. This is best visualized in FIGS. 5A and 8F. Moreover, the rounded spur 60 extends more than one-third but less than two-thirds of the length of the bevel 12 on the side opposite to the face of the bevel 12 along the longitudinal axis of the needle 11. The widest diameter of the distal aperture of the needle 11 (shown by the continuous vertical lines 80 in FIGS. 8A-F) defined by the non-bevel part 71 and the bevel part 70, is shorter than the widest diameter of the distal opening of the catheter 10 (shown by broken vertical lines 81 in FIGS. 8A-F) defined by the half oval opening 40 and the rounded spur 60.

The rounded spur 60 is meant to provide extra assurance that some part of the catheter 10 is inside the vessel lumen 30 whether the aperture 16 and the opening 40 are completely within the vessel lumen 30 or not. The rounded spur 60 also provides accommodating room for the tip 76 of the bevel 12 as it is withdrawn into the catheter 10 as shown in FIG. 6. Toward the proximal end, the needle 11 optionally has a colored marking 61 (e.g., blue, green or white) on its body, which indicates the exact position of the bevel 12 on withdrawal when it is just within the safe confines of the distal opening of the catheter 10. Also, advancing the cannula 50 in this position ensures that the catheter is not kinked or sheared by the sharp bevel 12 in case of a kink. During routine intravascular cannulation, if the needle 11 is pulled too far out of the catheter 10, the soft pliable plastic catheter 10 can easily become kinked on the slightest obstruction to the intravascular advancement resulting in shearing of the catheter 10 by the sharp bevel 12. By having a colored indicator 61 and following the method of the present invention as described following, intravascular placement is standardized to avoid the most common error contributing to technique failures.

In practice, the technique to place the preferred embodiment of the intravascular cannula 50 of the present invention would be as follows:

Step b 1: Insert and direct the cannula 50 slowly towards the targeted vessel after puncture of the skin by bevel 12.

Step 2: Stop moving the cannula 50 when flashback of blood is observed in the proximal hub base 14.

Step 3: Hold the catheter 10 in place and slightly withdraw the needle 11 backward.

Step 4: Stop withdrawing the needle 11 when the colored indicator 61 becomes visible.

Step 5: Advance the catheter 10 over the needle 11 into the vessel lumen 30. One can move only the catheter 10 while holding the needle 11 in place or by simply advancing the whole cannula assembly 50, until the catheter 10 has been inserted up to its proximal end.

Step 6: The needle 11 is completely withdrawn leaving the catheter 10 in the targeted vessel: a final step common to all intravascular cannulation.

U.S. Pat. No. 5,649,911 also describes an embodiment of a catheter-needle assembly with a side opening in the outer plastic cannula or catheter corresponding with a side hole adjacent to the sharp end of the inner metallic cannula or needle. This arrangement is intended to ensure simultaneous placement of the two openings in the vessel lumen on visual observation of blood in the flash chamber. The openings are in the shape of a complete circle which implies that the whole of the bevel length as well as the circular openings have to be within the blood vessel lumen for the flashback to occur. Since the intravascular cannula is entered into a blood vessel at a small angle, such an embodiment would result in counter-puncture of the back wall of the vessel almost invariably. Advancing the plastic cannula over the inner metallic cannula is this setting would result in catheter placement outside the blood vessel. The patent fails to specify or describe a method to overcome these problems.

These problems are addressed in embodiments of the present invention. A corresponding half oval opening at the bevel take-off point and a rounded spur that projects beyond distal aperture of the needle ensures that the catheter and the needle are simultaneously placed in the blood vessel lumen without having to pass the whole length of the bevel and the corresponding openings. The rounded spur ensures that some part of the catheter is already in the vessel lumen when flashback occurs in the situation of Possibility 2 and Possibility 3. By pulling back on the metallic needle to the colored marking, the possibility of counter-puncture and out-of-vessel placement is averted. Also, kinking is less likely to occur as only a pre-determined part of the needle is pulled out of the soft catheter during placement owing to the colored marking placed on the proximal side of the needle. A method of intravascular placement of an indwelling cannula is thereby standardized.

The detailed description and the accompanying drawings are not intended to limit the scope of the invention in any way. The inventor believes that the modified distal assembly of embodiments of the present invention can apply to any device used to penetrate body structures where flashback of some body fluid is used to indicate successful penetration of a body cavity or space. The scope of this invention is wide and can apply to following devices, methods, specialties or situations:

1. Intravenous peripheral indwelling cannulas, used by health care workers in all fields or specialties, all over the world.

2. Intravenous central cannula to access initial intravenous access prior to placement of a central venous catheter. It includes all Medical, Surgical specialties including Anesthesiology.

3. Intravenous peripherally inserted central catheter (PICC line) to achieve initial IV access.

4. An arterial indwelling catheter for direct invasive blood pressure monitoring and blood sampling. It is widely used in Anesthesiology and Critical Care Medicine.

5. A variety of both intravenous and intra-arterial catheters used by Vascular Surgeons, Interventional Radiologists and Cardiologists. All catheters require placement of an indwelling cannula as an initial step.

6. The present invention can also be extended to cannulas used to penetrate the chest wall to drain pleural fluid percutaneously.

7. The present invention can also be extended to cannulas used to penetrate the peritoneal cavity to drain peritoneal fluid percutaneously.

8. The present invention can also be extended to cannulas used by Urologist, Nephrologists or Interventional Radiologists to penetrate the urinary bladder, ureter or renal pelvis percutaneously.

9. The present invention can also be extended to insert lumbar drains percutaneously for CSF drainage by Neurosurgeons.

10. The present invention can also be extended to cannulas used for pericardiocentesis.

11. The present invention can also be extended to a cricthyroidotomy cannula which is used to secure emergency airways is case of failed intubation or failed ventilation. If the catheter is inadvertently inserted into the tracheal wall or paratracheal tissues (a situation equivalent to Possibility 2 and Possibility 3 in FIG. 7B and FIG. 7C), it may result in subcutaneous emphysema, pneumomediastinum and pneumothorax because of high pressure jet ventilation. These are known complications of the procedure, which may be avoided by the use of the present invention.

For devices described in paragraphs [0053] to [0058], many variations of the bevel design such as its nature (blunt or sharp), its angle or length already exist but the core concept of the relationship between the catheter 10 and the needle 11 in the distal assembly as described herein for the present invention could be applied to any of these devices to improve the initial cannulation success rate.

Most of the discussion in the above sections applies very much to the art of intravenous cannulation. For intra-arterial cannulation, the problem is even worse. The distal bevel assembly for currently available intra-arterial cannulas is the same as for intravenous cannulas. Intra-arterial cannulation should be no different from, or more difficult than, an intravenous cannulation, by general experience and majority consensus would state otherwise. The failure rate is much higher with intra-arterial cannulation, making this art an exclusive domain of a few practitioners. In the opinion of the present inventor, there are sound explanations for this kind of failure. As compared to the venous system, the arterial system is a very high pressure system. During placement of an intra-arterial cannula, the flashback of blood is very rapid, prompting the user to stop the needle insertion thinking that the distal tip is well inside the vessel lumen, though it may not be the case. In fact, due to very high pressure, the flashback would be observed even when only a fraction of the bevel and its aperture is inside the vessel, though the distal end of the catheter is possibly still outside the lumen. In the prior art of arterial cannula assembly, the catheter is shorter than the needle and ends proximal to the bevel take-off point just like the intravenous cannula. Due to high arterial pressure, the incidence of Possibility 2 and Possibility 3 is much higher leading to technique failure and multiple attempts causing undue distress to the patient. In the case of an arterial cannula, there have been attempts to correct this anomaly in order to improve simultaneous placement of the needle and the catheter in the vessel lumen. In an earlier version of an open arterial cannulation system, a metallic cannula is directly inserted into the arterial lumen. When pulsatile spurting of arterial blood is observed through the open hub of the needle, a guide-wire is inserted via the needle into the vessel (Seldinger technique). The needle is removed leaving the guide-wire in place and a plastic cannula (arterial catheter) is passed over the wire into the artery. The success rate was very high but the procedure was messy due to spurting of blood. This technique fell out of favor due to increased risk of HIV and Hepatitis with blood exposure.

In the next generation; e.g., the Arrow Radial Artery Catheterization system claims the following features: “an integral spring-wire guide technique; extra-short bevel on the introducer needle; the precision fit between the needle bevel heel and catheter tip that facilitates accurate placement within the vessel lumen; blood flashback in the clear hub of the introducer needle shows successful entry; feed tube allow easy visualization of blood flashback and inhibits blood exposure; actuating lever simplifies spring-wire guide and catheter advancement into vessel”.

In Arrow QuickFlash® Arterial Catheters, the technology is advanced one step further and they claim the following features: “Safer, more reliable arterial placements with virtually instantaneous sign of arterial access, Arrow QuickFlash® Arterial Catheters let you know when to stop needle insertion and help you avoid the accidental puncture of both arterial walls; as soon as you enter the artery, windows along the introducer needle immediately indicate blood flow through the transparent polyurethane catheter; patented spring-wire guide technique; extra-short needle bevel with precision fit between heel and catheter tip improves simultaneous placement of needle and catheter in vessel lumen; actuating lever simplifies spring-wire guide and catheter advancement into vessel; clear introducer needle and hub and plastic feed tube allow easy visualization of blood flashback”.

From the above discussion, it appears that technology is moving towards improving simultaneous placement of the needle and the catheter with a clear indication of when to stop the needle insertion. There have been many innovations in arterial cannulation devices, but surprisingly they have not been extended to intravenous cannulation. There has been little change in the basic design of an intravenous cannula other than some variations in designs of bevel, hub, side port or side wings. The distal assembly that describes the integral relationship between catheter and needle ends has changed little in the past 50 years.

It is easy to comprehend why innovations of arterial cannulas have not been extended to intravenous cannulas. Technology does not come cheap. Highly sophisticated designs like QuickFlash® will make intravenous cannulas very expensive. The intravenous cannula is probably the most common piece of equipment used in health care systems all over the world. Making IV cannulas an expensive commodity would have far reaching effects on overall economy of any health care facility. As health care workers, we take it for granted that failed attempts at IV cannulation are common and acceptable. Even the patient population has gotten used to the concept of “missed attempts” at IV cannulation.

The present inventor believes that we ought to change this mindset across the boar. There is a dire need to modify the design of a typical intravascular cannula not only for professional satisfaction but also for the patients' comfort. The modifications should be simple, practical and without any cost implications. The present invention achieves these objectives. The ultimate goal is to have one intravascular cannula, which can be used for both intravenous and intra-arterial access with equal success rate, in which “flashback” is a definite sign of perfect cannulation and success does not depend on the gauge of the cannula, and where there is standardization of the procedure with a consistent approach to the technique.

There are some limitations and disadvantages of embodiments of the present invention that may be eliminated by future research involving the preferred embodiments. Moving the distal aperture from the bevel to the bevel take-off point (the bevel heel) is demanding in that a longer length of the cannula tip has to be inside the vessel lumen for the flashback to be observed. That could increase the possibility of counter-puncture of the opposite wall of the vessel. The situation can be rectified by pulling the needle inside the catheter until the proximal colored marking is seen on the needle. In the prior art, the diameter of the internal lumen of the needle and its distal aperture increases with the gauge of the needle. The distal aperture with its hollow lumen in the needle is connected to a transparent hub base that gives visual indication of the “flashback”. That is the only purpose of the “aperture-lumen-hub” complex of the needle. The present inventor does not believe that we need to increase the internal diameter of the aperture and the needle lumen as we increase the size (gauge) of the intravascular cannula. The aperture and internal lumen diameter of a 20 Gauge IV cannula give a very good visual indication of the flashback and the same aperture and internal lumen diameter should work equally well for other larger gauge cannulas: 18 Gauge, 16 Gauge and 14 Gauge. In conclusion, we can standardize the diameter of the distal aperture and the needle lumen for IV cannulas for Gauges 20 and upward. In this way, we can minimize the dimension of the distal aperture and its overlying half oval opening, thereby decreasing the desired length of an IV cannula tip that has to be within the vessel lumen before visual indication of the flashback. For smaller gauge cannula used in Pediatric practice; i.e., 22G, 23G, 25G, this issue does not have any implication.

The present invention has been described with reference to certain preferred and alternative embodiments that are intended to be exemplary only and not limiting to the full scope of the present invention. In particular, while the preferred embodiments are described with respect to intravascular cannulation, the present invention is not so limited and may be applied more generally to cannulation through a lumen wall and into a lumen where flashback of a bodily fluid verifies insertion into the lumen.

While the foregoing is directed to embodiments of the present invention, other and further embodiments of the invention may be devised without departing from the basic scope thereof, and the scope thereof is determined by the claims that follow.

1. A cannula assembly comprising:
an inner cannula for puncturing a body structure, the inner cannula comprising:
a proximal end having a proximal aperture, and
a distal end comprising:
a bevel for facilitating puncturing of the body structure,
a distal aperture disposed partially within the bevel so that the distal aperture includes a beveled portion and a non-beveled portion, a bevel take-off separating the beveled portion and the non-beveled portion,
wherein the distal aperture and proximal aperture are connected through a hollow internal passageway through the inner cannula; and
an outer cannula for placement within a cavity of the body structure to provide a fluid path to and from the cavity, the outer cannula having a distal end and a proximal end,
wherein the inner cannula is generally concentrically disposed within the outer cannula and the inner and outer cannula are slideable relative to one another.

2. The cannula assembly of claim 1, wherein the non-beveled portion of the distal aperture is disposed adjacent to the bevel take-off in a generally tubular wall of a body of the inner cannula, the body of the inner cannula disposed between the bevel and the proximal end, and wherein the beveled portion is disposed distally adjacent to the bevel take-off in the bevel.

3. The cannula assembly of claim 1, wherein the non-beveled portion of the distal aperture is generally half-oval shaped, the center of the generally half-oval at or near the bevel take-off.

4. The cannula assembly of claim 1, wherein the beveled portion of the distal aperture comprises approximately ⅓ or less of the bevel.

5. The cannula assembly of claim 1, wherein the outer cannula comprises:
a distal opening at its distal end; and
a proximal opening at its proximal end, the distal opening and proximal opening connected to one another via a hollow internal passageway through a wall of the outer cannula,
a portion of the distal end of the wall of the outer cannula having an extending opening therein which extends from the distal opening, the extending opening corresponding in shape to and capable of overlying the non-beveled portion of the distal aperture to communicate therewith when the distal end of the outer cannula is located within the cavity of the body structure.

6. The cannula assembly of claim 5, wherein the extending opening is generally half-oval shaped to correspond with a generally half-oval shaped non-beveled portion of the distal aperture of the inner cannula.

7. The cannula assembly of claim 5, wherein an indicating member disposed at or near proximal ends of the inner and outer cannulas is capable of indicating axial and circumferential alignment of the corresponding extending opening and non-beveled portion of the distal aperture.

8. The cannula assembly of claim 5, wherein the outer cannula further comprises a spur extending from a distal end of the outer cannula generally circumferentially opposite from the extending opening, the spur capably of ensuring that some portion of the outer cannula is within the cavity of the body structure at the moment of “flashback”.

9. The cannula assembly of claim 1, further comprising a chamber operatively connected to the proximal end of the inner cannula for holding a flashback of body fluid therein, the body fluid flowed through the hollow internal passageway from the lumen of the body structure.

10. The cannula assembly of claim 1, further comprising a Luer lock mechanism operatively connected to the proximal end of the outer cannula.

11. The cannula assembly of claim 1, wherein the inner cannula further comprises a marking on an outer portion of its body towards proximal end to indicate the position of the bevel when it is within the confines of the distal opening of the outer cannula.

12. The cannula assembly of claim 1, wherein approximately ⅔ or more of the distal end of the inner cannula is non-cannulated, serving only to puncture skin and the body structure.

13. The cannula assembly of claim 1, wherein the body structure is a blood vessel and fluid within the body structure is blood.

14. The cannula assembly of claim 1, wherein the outer cannula is shorter than the inner cannula and the distal end of the outer cannula terminates at the bevel take-off.

15. A method of accessing a cavity of a body structure using a cannula assembly, comprising:
providing the cannula assembly comprising:
an inner cannula having a beveled tip at its distal end and an aperture therein having a beveled portion disposed across a portion of the beveled tip and a non-beveled portion, the aperture extending through a body of the inner cannula to its proximal end, and
an outer cannula having an internal hollow passageway therethrough and an opening within a portion of its wall at its distal end, the opening capable of overlying and generally corresponding with the non-beveled portion of the inner cannula distal aperture;
puncturing the wall of the body structure using the beveled tip by moving the cannula assembly towards the body structure;
upon observing flashback of bodily fluid into a chamber of the cannula assembly, halting moving of the cannula assembly;
withdrawing the inner cannula backward relative to the body structure until the colored marking indicates that the inner cannula is within the confines of the outer cannula;
advancing the outer cannula into the cavity within the body structure; and
withdrawing the inner cannula while leaving the outer cannula in the wall of the body structure.

16. The method of claim 15, further comprising advancing the outer cannula over the inner cannula when advancing the outer cannula into the cavity within the body structure.

17. A cannula assembly comprising:
an outer cannula having a distal opening and a proximal opening therein which are connected via an internal hollow passageway through the outer cannula, the distal opening being on the insertion end of the outer cannula and having uneven margins defined by a generally half-oval defect and a spur; and
an inner cannula having a sharp, beveled distal end and a proximal end opening into a flash chamber, the distal end and proximal end connected via an internal hollow passageway through the inner cannula, the distal end having an aperture therein at or near the bevel take-off point dividing the aperture into a beveled portion and a non-beveled portion,
the inner cannula received coaxially within the internal hollow passageway of the outer cannula so that the generally half-oval defect of the outer cannula corresponds with the non-beveled portion of the distal aperture of the inner cannula.

18. The cannula assembly of claim 17, wherein the proximal opening of the outer cannula comprises a locking mechanism for connection to intravascular fluid-giving set tubing and a ridge or wing indicating a position of the beveled distal end.

19. The cannula assembly of claim 17, wherein an alignment means for indicating when the generally half-oval defect of the outer cannula corresponds with the beveled portion of the distal aperture of the inner cannula is operatively connected at or near a proximal end of the cannula assembly.

20. The cannula assembly of claim 17, wherein the beveled portion of the aperture of the inner cannula comprises only a portion of the beveled distal end of the inner cannula.
